(19) United States
(12) Patent Application Publication
(10) Pub. No.: US 2002/0068295 A1
(43) Pub. Date: Jun. 6, 2002

(54) Multimeric biopolymers as structural elements and sensors and actuators in microsystems

(76) Inventors: Marc Madou, San Diego, CA (US); Leonidas G. Bachas, Lexington, KY (US); Sylvia Daunert, Lexington, KY (US)

Correspondence Address: CALFEE HALTER & GRISWOLD, LLP, 800 SUPERIOR AVENUE, SUITE 1400, CLEVELAND, OH, 44114, US

(21) Appl. No.: 09/905,041

(22) Filed: Jul. 13, 2001

Related U.S. Application Data
(60) Provisional application No. 60/218,036, filed on Jul. 13, 2000.

(51) Int. Cl. C12Q 1/68; G01N 33/53; C12N 9/16; C07K 14/00

(52) U.S. Cl. 435/6; 435/7.1; 530/350; 435/196; 530/395

(57) ABSTRACT

Biomolecular complexes, hereinafter referred to a mulimeric biopolymers which can be used as the foundation of chemical control systems capable of both sensing the presence of a target analyte and actuating some mechanical response. The biomolecular complexes are multimeric biopolymers comprising at least two monomeric units. The monomeric units are selected from the group consisting of full-length proteins, polypeptides, nucleic acid molecules, and peptide nucleic acids. At least one of the monomeric units binds to the target analyte. In one highly preferred embodiment the multimeric biopolymers of the present invention undergo a detectable conformational change in response to exposure to an analyte. The present invention also provides micromachined and nanomachined devices and systems which employ the multimeric biopolymers to sense the presence of a target analyte, to actuate a response to the presence of a target analyte, or to perform both functions.

This application claims priority to U.S. Provisional Application Serial No. 60/218036, filed Jul. 13, 2000.

The work described in this application was supported, at least in part, by ______ Grant No. ______. The U.S. Government has certain rights in the inventions.

### BACKGROUND

Proteins and DNA are information rich molecules with structural and electrical properties which make their incorporation into the human manufacturing arsenal an attractive proposition. Several microstructures using oligonucleotides as building blocs have been demonstrated (N. C. Seeman, Ann. Rev. Biophys. Biomol. Struc., vol. 27, pp. 225, 1998; E. Winfree, F. Liu, L. Wenzler, and N. C. Seeman, Nature, vol. 394, pp. 539, 1998.), and many particles/objects have been derivatized with DNA strands or oligonucleotides (R. Bashir, “DNA-Mediated Artificial Nano-Bio-Structures: State of the Art Future Directions,” Superlattice and Microstructures, vol. 29, pp. 1-16, 2001.) Short strands of DNA, also known as aptamers have been suggested as a tool in DNA mediated self assembly of micro components into larger subassemblies or onto a PC board (C. F. Edman, C. Gurtner, R. E. Formosa, J. J. Coleman, and M. J. Heller, “Electric-Field-Directed Pick-and-Place Assembly,” HDI, vol. October, pp. 30-35, 2000; C. F. Edman, R. B. Swint, C. Gurtner, R. E. Formosa, S. D. Roh, K. E. Lee, P. D. Swanson, D. E. Ackley, J. J. Coleman, and M. J. Heller, “Electric Field Directed Assembly of an InGaAs LED onto Silicon Circuitry,” IEEE Photonics Tech. Lett., vol. 12, pp. 1198-1200, 2000; C. A. Mirkin, R. L. Letsinger, R. C. Mucic, and J. J. Storhoff, Nature, vol. 382, pp. 607, 1996.). Proteins have also been used in a wide variety of microstructures with motor proteins perhaps the most studied example. The first examples of combinations of proteins with micromachined structures were realized recently. One example of this hybrid human/natural manufacturing trend is a Ni rotor blade affixed to a motor protein (Montemagno at Cornell, http://www.sciam.com/explorations/2000/112700nano/).

The combination of the natural biopolymers with microelectromechanical systems (MEMS) and nanoelectromechanical systems (NEMS) promises the advent of a totally new class of sensors and actuators with applications in drug delivery, diagnostics, biocompatible surfaces, prosthetics and many other fields.

### SUMMARY OF THE INVENTION

The present invention provides biomolecular complexes, hereinafter referred to a mulimeric biopolymers which can be used as the foundation of chemical control systems capable of both sensing the presence of a target analyte and actuating some mechanical response. The biomolecular complexes are multimeric biopolymers comprising at least two monomeric units. The monomeric units are selected from the group consisting of full-length proteins, polypptides, nucleic acid molecules, and peptide nucleic acids. At least one of the monomeric units binds to the target analyte. In one highly preferred embodiment the multimeric biopolymers of the present invention undergo a detectable conformational change in response to exposure to an analyte.

The present invention also provides micromachined and nanomachined devices and systems which employ the multimeric biopolymers to sense the presence of a target analyte, to actuate a response to the presence of a target analyte, or to perform both functions. In one highly preferred embodiment, the device comprises a substrate having at least one storage chamber which contains a substance which is released therefrom when the multimeric biopolymer undergoes a change in its three dimensional conformation.

In general, such devices and systems involve integration of the multimeric biopolymers into MEMS and NEMS devices, where chemical control of a given device may be complemented by electrical control to ensure maximum safety and efficacy in use of the device.

The present invention also relates to methods of using the devices and systems of the present invention to dispense a substance in response to binding of the analyte to the multimeric biopolymer.

### BRIEF DESCRIPTION OF THE FIGURES

The present invention may be more readily understood by reference to the following drawings wherein:

FIG. 1 is an illustration of calmodulin undergoing a conformational change when it binds calmodulin and the subsequent binding of phenothiazine to calmodulin; and

FIG. 2 is an illustration of hydrogel deposited on a metal electrode (e.g., Pt) as an actuator, showing water hydrolysis and reversible swelling and shrinking of the hydrogel; and

FIG. 3 is an illustration of an example of polymer proteins functioning as sensors/actuators; and

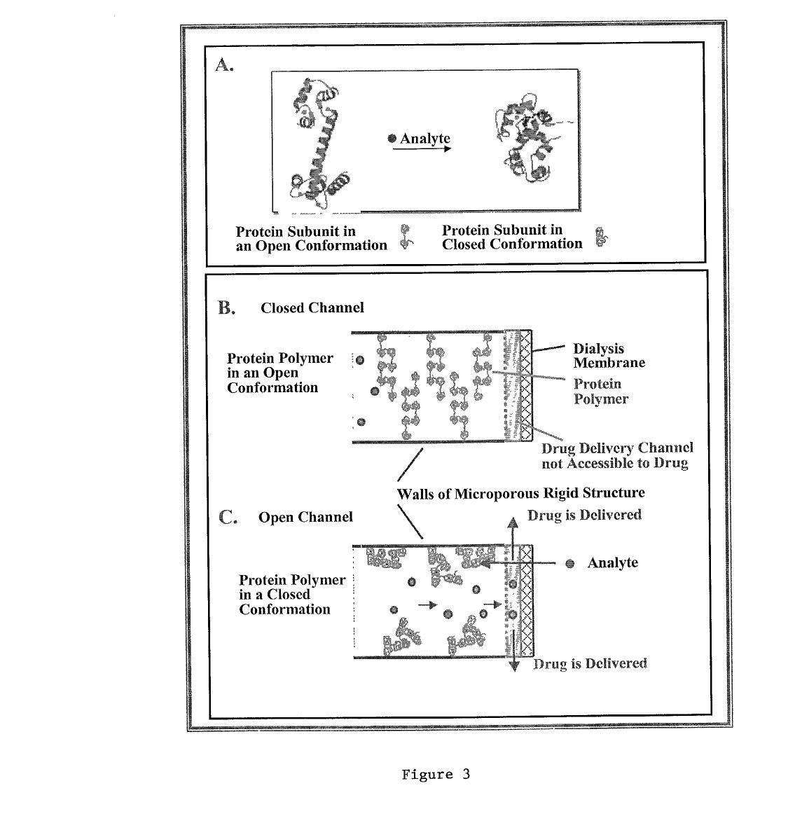

FIG. 4 is an illustration of wiring a multimeric biopolymer (DNA in this case) with a redox polymer to an underlying conductive microelectrode where the DNA is anchored to the redox material bia a biotin-streptavidin linkage.

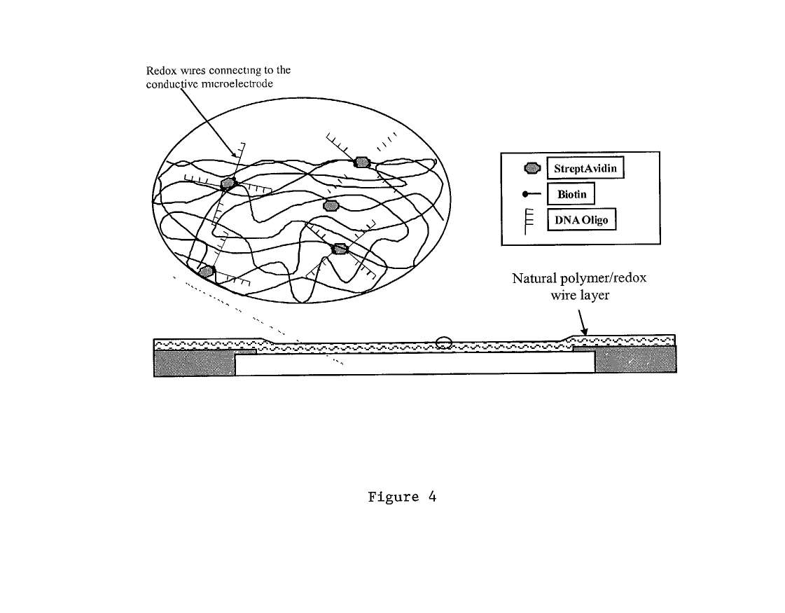

### DETAILED DESCRIPTION OF THE INVENTION

In one aspect the present invention provides new biocomplexes which can be used to sense the presence of an analyte, to actuate a mechanical response when exposed to an analyte, or to perform both functions. The biomolecular complexes of the present invention are multimeric biopolymers.

In another aspect, the present invention also provides micromechanical devices and biosensors, particularly MEMS and NEMS, which contain the multimeric biopolymers of the present invention. In one embodiment, the device further comprises a hydrogel. In another embodiment, the device further comprises a redox polymer. In another embodiment, the device further comprises both a redox polymer and a hydrogel.

As used herein the term “sensor” refers to a multimeric biopolymer which gives off a detectable signal, such as for example, a fluorescent signal in response to an analyte.

As used herein the term “actuator” refers to a multimeric biopolymer that (a) exhibits a mechanical response when exposed to an analyte or (b) causes another substance, such as for example a hydrogel, to exhibit a mechanical response when the multimeric biopolymer is exposed to an analyte.

In the industry the terms MEMS and NEMS refer to (Microelectrochemical systems and Nanoelectrochemical systems) i.e., systems that comprise a machined microstructure or nanostructure, respectively, such as for example a chip comprising a polysilicon membrane for pressure sensing. Such systems further comprise an electronic component which may either be part of the microstructure or nanostructure or in hybrid fashion coupled thereto.

Multimeric Biopolymer

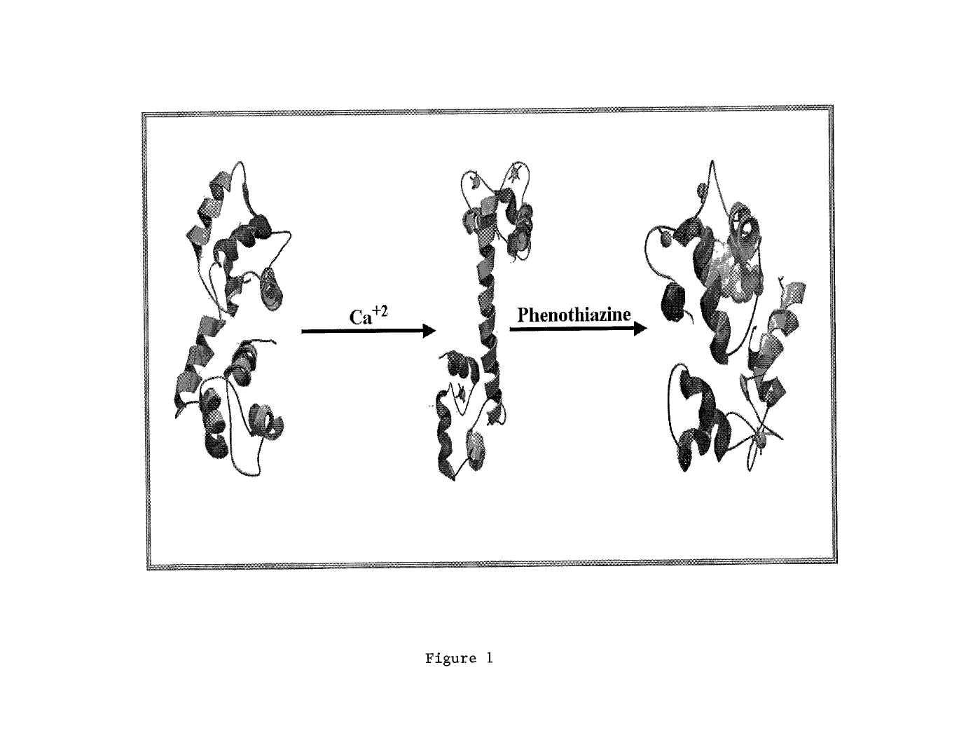

As used herein the term “biopolymer” refers to a biomolecule capable of responding to a change in its microenvironment. Examples of biopolymers are proteins, polypeptides, and nucleic acid molecules. One way in which a biopolymer can respond to a change in its microenvironment is by changing its conformation. For example, one way in which a protein can change conformation is by unfolding, totally or in part (i.e., local areas of the protein can unfold). Examples of microenvironmental changes that can cause the biopolymers to respond include such things as an increase or decrease in pH or an increase or decrease in the concentration of specific analyte(s). One specific example of a biopolymer is calmodulin. The specific analyte bound by calmodulin are calcium ions and the anti-psychotic phenothiazine class of drugs. Calmodulin molecules respond to binding calcium by changing conformation (FIG. 1). In addition, when phenothiazines are present, calmodulin responds by undergoing additional change in conformatin.

In one aspect, the present invention provides a synthetic multimeric biopolymer that comprises at least two, preferably a plurality, of monomeric units of a biopolymer. At least one of the monomeric units, and preferably a plurality of the monomeric units, comprise one or more binding regions that bind to an analyte. The analyte may be a biochemical that is found in an organism (e.g., bacteria, yeast, animals, humans, plants, etc.), such as for example a sugar, a protein, a nucleic acid, a hormone, a vitamin, or a co-factor. The analyte may also be an ion such as for example a hydrogen ion, a hydroxyl ion, an oxyanion (e.g., phosphate, sulfate, etc.) or a cation (e.g., calcium ion, etc.). The bonds that form between the analyte and the binding region include all chemical bonds except covalent bonds. Examples of such chemical bonds are ionic bonds, hydrogen bonds, hydrophobic interactions and van der Walls forces.

The monomeric unit is selected from the group consisting of a full-length protein, a polypeptide which is a fragment of a full-length protein, a nucleic acid molecule, which is preferably an aptamer, a peptide nucleic acid. The monomeric units may be the same or different.

In one highly preferred embodiment the multimeric polymer undergoes a detectable conformational change in response to exposure to the analyte. Such a composition is a structurally linked multimer of biomolecules (e.g., multimers composed of linked proteins, DNA, RNA, peptide nucleic acids, etc.), and combinations thereof. When disposed within a device, such as for example a polymeric drug delivery device or a machined microstructure or nanostructure, the conformationally-reactive multimeric biopolymer can be used to open or close a channel, either directly or indirectly. As used herein, this response to the analyte is referred to as an actuating event.

In another embodiment, exposure of the multimeric biopolymer to the analyte causes the multimeric biopolymer to emit a detectable signal, such as for example a fluorescent signal. Examples of such detectable signals are fluorescent signals, an optical signals, electrochemical signals, pressure changes, changes in dielectric constant, mass changes, volume changes, and temperature changes. Such multimeric biopolymers can be used as a sensor, particularly within a MEMS or NEMS to detect the presence of the analyte and to generate a signal which is transmitted to a transducer.

One example of a multimeric biopolymer of the present invention is a dimer of the calmodulin protein. The calmodulin dimer comprises a protein where the C-terminal end of one calmodulin molecule is attached to the N-terminal end of an adjacent calmodulin molecule. Calmodulin undergoes a hinge-type motion upon binding to calcium. Its crystal structure has been well-studied using X-ray crystallography and NMR techniques. Calmodulin consists of two domains, the N- and the C-domain. Two high affinity calcium-binding sites are located in the C-domain and the other two low affinity calcium-binding sites are located in the N-domain. Upon binding to calcium, calmodulin undergoes a change in conformation, which exposes two hydrophobic pockets located in the N -and C-domains (FIG. 1). Certain hydrophobic peptides and the anti-psychotic phenothiazine class of drugs interact with these exposed hydrophobic pockets. Another example of a multimeric biopolymer of the present invention is a polymer comprised of glucose or galactose binding proteins.

Like the monomeric units, the multimeric biopolymers change their conformation in response to the microenvironment. In fact, changes in multimeric biopolymers in response to a particular microenvirnomental change are greater in magnitude than are changes in monomeric units that comprise the multimeric biopolymer that are caused by the same microenvironmental change. For example, the conformational change induced in the calmodulin dimer is greater in magnitude than the conformational change induced in a separately tested, single calmodulin molecule in response to calcium binding. Such changes in multimeric biopolymers, therefore, can be additive or even greater than additive, compared to the changes in the monomeric units that comprise the biopolymer, in response to the same microenvironment.

Multimeric Proteins and Polypeptides

The multimeric proteins and polypeptides of the present invention comprise at least two, preferably from 2 to 10 proteins or polypeptides. At least one, preferably a plurality, of the monomeric units of the multimeric protein comprise a binding region for an analyte. The monomeric units of the multimeric proteins and polypeptides may be the same or different. For example, the multimeric protein may be comprised of a single protein. Alternatively, the multimeric protein may comprise a structural protein which changes its conformation in response to contact with an analyte and an enzyme which catalyzes a chemical reaction with its specific substrate. Catalysis of such reaction results in release of protons or removal protons from the microenvironment of the multimeric protein.

In certain instances, the conformationally-reactive multimeric proteins of the present invention are designed to undergo a change in response to binding of a specific biochemical to the binding site or sites in the multimeric protein. In other instances, the conformationally-reactive multimeric proteins of the present invention are designed to undergo a change in conformation in response to a change in ion concentration, particularly a change in hydrogen ion or hydroxide concentration. For example, ion concentration changes above or below the isolectric point of the protein will cause the protein to change its three-dimensional shape.

The multimeric proteins may comprise a plurality of one or more structural proteins that undergo a conformational change in response to binding to an analyte. Alternatively, the multimeric proteins may comprise a plurality of enzymes linked to or in close proximity to a plurality of structural proteins. Upon binding to their respective substrates, the enzymes catalyze a reaction that leads to a change in pH in the microenvironment surrounding the structural protein thereby causing a change in conformation of the structural proteins.

Methods for preparing multimers of proteins are known in the art and a variety of methods exist. In one method, sulfhydryl groups present in cysteine amino acids of different proteins are used to create covalent bonds between the separate proteins. This is done through formation of disulfide bonds between the cysteines in the different proteins. Such disulfide bond formation occurs under oxidative conditions, i.e., atmospheric oxygen catalyzes formation of the disulfide bonds. In using this method of forming protein multimers, care must be taken to ensure that the cysteines involved in formation of the disulfide bonds will not affect the structure or function of the protein in an adverse way.

In addition to crosslinking through disulfide bond formation, other methods of chemical crosslinking of proteins to one another exist. For example, this can be achieved by either using directly reactive groups on the protein (e.g., amines, carboxylic groups, etc.) or by creating reactive groups on the protein (e.g., in the case of glycosylated proteins the sugars are oxidized to from aldehydes, acids, etc.). Once reactive groups on the protein exist, then they are directly reacted with the next protein or they are connected to the next protein via commercially available mono- or bifunctional linkers by following well-established protocols.

Other approaches to making protein multimers involve manipulation of the genes encoding such proteins. After manipulation, the genes are used to produce the proteins. Such proteins may be multimeric proteins or may be proteins that are then crosslinked to one another, as described above.

For example, in one instance, genes encoding proteins (the same protein or different ones, depending on the chosen application) can be fused together, end-to-end or start-to-end from their N- and C-termini, using recombinant DNA techniques. In such method, plasmids are constructed that incorporate the gene of the designed chosen multimer protein. The plasmids are inserted into bacterial, yeast, or mammalian vectors. The proteins are then expressed and purified using established molecular biology protocols.

Such recombinant DNA techniques can also be used to produce the monomeric subunits of what is to become the protein multimer. In such method, site-directed mutagenesis is used to remove or create unique amino acids in the protein monomer that facilitate attachment of one protein to another. Such site-directed mutagenesis techniques are well known to those skilled in the art. For example, such method can be used to introduce cysteine amino acids into the protein monomers. When the manipulated gene is then used to produce the proteins, such proteins can then readily be crosslinked to one another, as described above. Such techniques are described in U.S. Pat. Nos. 4,132,746 and 4,187,852.

Conformational changes in multimeric proteins can be detected using techniques such as NMR and X-ray crystallography. Several biosensing systems have been developed in which a fluorophore is attached to a unique site in the protein (Salins, L. L. E., Schauer-Vukasinovic, V., Daunert, S. SPIE-Int. Soc. Opt. Eng. 1998, 3115 16-24; Schauer-Vukasinovic, V. Cullen, L., Daunert, S. J Am. Chem. Soc., 1997, 119, 11102-11103; Wenner, B. R. Douglass, P. M., Shrestha, S., Sharma, B. V., Lai, S., Madou, M. J., Daunert, S. Proceedings of SPIE, 2, 59-70, 2001; L. L. E. Salins, C. Mark Ensor, R. Ware, and S. Daunert, Anal. Biochem., in press, 2001). The change in conformation in the presence of a ligand is then monitored by measuring a change in fluorescence of the reporter fluorophore.

The multimeric proteins of the present invention are dimers, trimers, and multimers of the same protein or of combinations of two or more different proteins forming a polymer. The genetically engineered polymer proteins can be used as sensors/actuators in a variety of applications that range from biosensors to responsive drug delivery systems to molecular machines. Therefore, we envision applications in environmental analysis, and in the diagnostics, biotechnology, and pharmaceutical industries.

Multmeric Nucleic Acids

The multimeric biopolymers of the present invention can also be nucleic acid molecules, such as DNA or RNA. As for multimeric proteins, described above, the nucleic acid multimers comprise repeating units of two or more smaller, monomeric molecules. Such monomeric units may be the same or different. Such monomers, as well as the multimeric nucleic acid, are able to respond to the presence of an analyte.

One such type of nucleic acid monomer that can be used to make multimeric nucleic acids is called an oligonucleotide ligand or “aptamer.” Aptamers are single-stranded DNA or RNA molecules that bind with high affinity to specific target or analyte molecules. Such analyte molecules can be drugs, vitamins, hormones, antibodies, enzymes, co-factors, nucleotides, proteins and so forth. Aptamers can range from between 8 to 120 or more nucleotides in length. Within this nucleotide sequence is contained a minimal sequence needed for binding to the analyte. Such sequence is normally between 15 to 50 nucleotides in length. Aptamers undergo a conformational change after binding of specific analytes. The binding constant of aptamers to their specific analyte molecules ranges from micromolar to sub-nanomolar ranges.

Aptamers have a number of advantages over other molecules that specifically bind target molecules. Aptamers have remarkable specificity for their specific analytes. Aptamers can discriminate between analytes based on subtle differences in the analytes. For example, aptamers can discriminate between analytes based on the presence or absence of a methyl or hydroxyl group. Aptamers can discriminate between analytes based on the difference between the D- and L-enantiomer.

Another advantage of aptamers is that their synthesis is straightforward. Aptamers are produced by chemical synthesis, which is extremely accurate and reproducible. Aptamers produced by such synthesis can be purified, under denaturing conditions, to a high degree. Reporter molecules, fluorophores for example, can subsequently be easily attached to purified aptamers. Such attached fluorophores can emit a fluorescence signal whose intensity varies depending on whether the aptamer has or has not bound its target analyte. Such differential emission of fluorescence in response to target binding can facilitate the use of such labeled aptamers as sensors and actuators.

Aptamers that bind selectively to a specific analyte are commonly selected from very large random sequence oligonucleotide libraries comprised of as many as 10¹⁵ random sequences (McGown, et al., 1995, Anal Chem, 67:663A-68A; Jayasena, 1999, Clin Chem, 45:1628-50). Such selection involves an iterative enrichment process. Such process is called SELEX (systematic evolution of ligands by exponential enrichment). Steps in the SELEX process involve passing the entire oligonucleotide library over a support, such as an affinity column, to which the analyte molecule is attached. The oligonucleotides that do not bind to the analyte in the column pass through the column and are discarded. The oligonucleotides that bind to the analyte are then eluted from the column. The oligonucleotides that elute from the column are then amplified using the polymerase chain reaction (PCR). The PCR-amplified pool of oligonucleotides is then passed over the column again, as described above, and the eluate is again amplified by PCR. The cycle is repeated numerous times. Commonly, the cycle is repeated anywhere from between 8 to 15 times.

Once aptamers are obtained, polymers of the aptamers are prepared. Such polymeric aptamers can be prepared by employing several strategies. For example, DNA synthesizers can be used to prepare a DNA segment that terminates in a functional chemical group (e.g., thiol, biotin, etc.). This allows for coupling of the DNA aptamer unit to form dimers, trimers, etc. of the original aptamer.

In another embodiment, the conformationally reactive multimeric biopolymers are aptamers, which are nucleic acid ligands composed of single strands of DNA or RNA. These are molecular recognition elements that upon binding to their respective ligands (e.g., drugs, vitamins, hormones, antibodies, enzymes, biological co-factors, etc.) undergo a conformational change (Jasayena, 1999; McGown et al, 1995; Jhavery et al., 2000). The binding constant of aptamers to their respective ligands ranges from μM to sub-nM (Hamassaki et al, 1998; Lee and Walt, 2000; Potyrailo, 1998), making them suitable for detection of biomolecules in biological fluids. The three-dimensional structure of a functional aptamer can be denaturated by temperature, pH, salt gradient, metal ions, and electrochemical potential (Jasayena, 1999). Thus, aptamers can be used in a similar fashion to the binding proteins mentioned above. Specifically, polymeric aptamers can be prepared by employing several strategies. For example, DNA synthesizers can be used to prepare a DNA segment that terminates in a functional chemical group (e.g., thiol, biotin, etc.). This allows for coupling of the DNA aptamer unit to form dimers, trimers, etc. of the original aptamer. Thiol-terminated aptamers can be coupled to each other by formation of disulfide bonds (connecting unit between two aptamers) under oxidizing conditions. In the case of biotin-terminated aptamers the connecting units can be avidin, streptavidin, or anti-biotin antibodies, for example. Avidin or streptavidin bind to up to four biotinylated compounds, which allows for organization of the aptamers in networks that are three-dimensionally different from those assembled by employing antibodies as connectors. Polymeric RNA aptamers can be prepared in a similar fashion.

Hydrogels

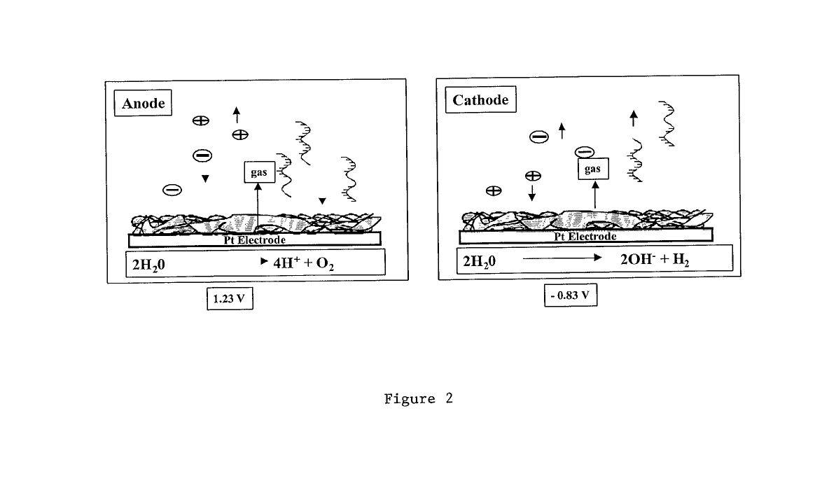

Hydrogels are networks of hydrophilic homopolymers or copolymers that exhibit dramatic effects of swelling and shrinking upon a stimulus. One such stimulus is movement or conformational change of the multimeric biopolymers. Another type of stimulus occurs when there is a change in pH in the environment in which the hydrogel is present. Such local pH change causes water and counter-ions to move in or out of the hydrogel and this induces swelling or shrinking of the hydrogel. This process is illustrated in FIG. 2 where a metal electrode underneath a hydrogel, causes hydrolysis and a local pH change.

Certain types of hydrogels undergo abrupt changes in volume in response to changes in pH, temperature, electric fields, saccharides, antigens and solvent composition. Natural and artificial hydrogels may also be forced to shrink or swell by applying a bias on a metal electrode underneath or embedded in a hydrogel gel. The process is illustrated in FIG. 2 for the case of a hydrogel on top of a Pt electrode. The hydrolysis process creates a local pH change, which changes the volume of the hydrogel. In this case the hydrogel acts an ionic type actuator, i.e., the polymer does not conduct electrons and actuation is induced by ion migration (somewhat similar to the way an action potential in a nerve cell is generated). The local pH change leads to a different charge on the polymer backbone and this causes water and counter-ions to move in or out of the hydrogel bulk and this, in turn, induces swelling or shrinking of the hydrogel. Depending on the type of hydrogel, a pH increase or pH decrease may induce the hydrogel volume changes. With the metal electrode used as an anode the pH decreases, and with the electrode used as a cathode the pH increases. This swelling behavior is governed by the amount of cross-linking of the hydrogel and the affinity of the polymer chains for solvent.

One type of hydrogel is an acrylamide or polyacrylamide (PA). It may be prepared by combining specific volumes of a filtered 40 wt % acrylamide solution, a 2 wt % N,N-methylenebisacrylamide solution, and a 98 wt % 2-(dimethylamino) ethyl methacrylated (DMAEMA) solution. The mixture may be deoxygenated by bubbling N₂ through the mixture for 15 minutes. A volume of 10-20 μl of a 10 wt % potassium persulfate solution may then be added to initiate the polymerization reaction.

A second type of hydrogel may be hydroxyethyl methacrylate (HEMA) based. A HEMA based hydrogel may be P(HEMA-co-MMA) and may be prepared by combining a co-monomer feed of 75 mol % HEMA and 25 mol % MMA, with 1 mol % ethylene glycol dimethacrylate (EGDMA) as the cross-linking agent and a trace amount of dimethoxy phenyl acetophenone (DMPA) as the photoinitiator. The polymerizations are carried out at ambient conditions. Three different compositions of PHEMA-DMAEMA may be prepared and tested. The first may consist of 0.198 HEMA, 0.0494 DMAEMA, and 0.0752 H₂O. The second may be composed of 0.198 HEMA, 0.0494 DMAEMA, 0.00220 EGDMA, 0.450 H₂O and 0.300 ethylene glycol. The compositions above are all in volume fractions. The third PHEMA-DMAEMA composition may be 76 wt % HEMA, 10 wt % DMAEMA, 2 wt % EGDMA, 12 wt % H₂O and a trace amount of DMPA.

In one embodiment of the present invention, hydrogels are placed in close proximity to the multimeric biopolymers, or are blended with multimeric biopolymers, in such a way that the stimulus for swelling or shrinking of the hydrogel is provided by the associated multimeric biopolymer when such biopolymer binds to its specific analyte. In this embodiment, the stimulus that causes swelling or shrinking of the hydrogel is the movement or conformational changes that occur in the multimeric biopolymer. In this case, the multimeric biopolymer directly causes the swelling or shrinking of the hydrogel.

In another embodiment of the present invention, binding of an analyte by the multimeric biopolymer results in release or consumption of protons. Such protons cause a local change in the pH and cause swelling or shrinking of the hydrogel due to movement of water and counter-ions into or out of the hydrogel, as described above.

Redox Polymers

The multimeric biopolymers of the present invention are most useful if the changes (e.g., conformational change) that they undergo in response to the microenvironment (e.g., binding of an analyte) are reversible. Reversibility allows the inventions of which the multimeric biopolymers are a component to be used more than once. That is, once the multimeric biopolymer binds its specific analyte and, for example, causes swelling and shrinking of a hydrogel, it would be advantageous if the multimeric biopolymer could be returned to its original state, for example the state in which no analyte is bound by the multimeric biopolymer.

One way in which the changes the multimeric biopolymers undergo can be reversed is through the use of redox polymers. Redox polymers are polymers, such as polypyrrole, polyaniline (PANI), polythiophene and the like, that are sensitive to pH, applied potential and chemical potential in their microenvironment. The redox polymers of the present invention are electronically conducting polymers. Such redox polymers, can conduct a current that originates from an electrode, for example, and when the redox polymer is in contact or close proximity to the mutimeric biopolymers, can reverse the changes that occurred in the multimeric biopolymer, by analyte binding, for example. In such case, the invention can be viewed as a “molecular gate” wherein the multimeric biopolymer opens or closes in response to analyte binding and wherein the redox polymer acts to override this process.

In another aspect, the present invention provides a device which employs the multimeric biopolymer as a molecular gate or actuator to regulate the flow of molecules, such as drugs, heparin, bioactivators, and ions through a channel or an opening in the device.

An example of the manner in which conformational changes of multimeric biopolymers may be utilized in conjunction with MEMS and NEMS is that of the incorporation of the multimeric biopolymers within channels of a substrate. These channels could, for example, be connected to a drug delivery chamber on one side. Opening and closing of the channels is accomplished by changing the conformation of the multimeric polymers. For example, in those cases where the biopolymer contains ligand-binding proteins (examples include binding proteins, receptors, enzymes, etc.), the conformational change occurs when the ligand binds to the protein.

The multimeric biopolymer may be attached to the channel surface, for example by a covalent bond. Alternatively, the multimeric biopolymer may be in a solution or suspension which is disposed within the porous substrate. Depending on the conformation of the biopolymer, the pores will be open or closed.

The device may be a MEMS or NEMS structure. Such structures are top-down machined devices with dimensions in the micrometer respectively nanometer range. They typically involve semiconductor industry type manufacturing methods. Products include pressure sensors, valves, pumps, accelerometers, gyros, . . . etc. With the ever decreasing dimensions of the lithography written features there is now an overlap between features that can be made with top-down methods and bottom-up methods (the ones described above to make the multimeric biopolymer sensors/actuators). This size overlap presents many new product opportunities. For example MEMS and NEMS structures may be manipulated by multimeric biopolymers. In such an embodiment, the multimeric biopolymer directly opens and closes the channel.

In a further embodiment the multimeric biopolymer is attached to or in communication with a movable door that is comprised of a rigid substance, such as for example silicon, or a hydrogel. The change in conformation that is initiated by binding of the analyte to the multimeric biopolymer causes the door to move, thereby opening or closing the channel. Such devices may further comprise a redox polymer which is blended with the multimeric biopolymer as described below.

Once the polymer proteins are prepared, they, preferably are coupled to the surface of the substrate. As is the case with the attachment of oligonucleotides to these surfaces, there are numerous well-established protocols for the successful attachment of proteins to surfaces (Rao, Anderson, Bachas, 1998- Full reference is typed at the end of the document). To limit loss of function or collapse of the three-dimensional structure of the multimeric biopolymer, hydrophilic surfaces are chosen. Inventors, how do your take a silicon, alumina or TiO2 substrate and make it hydrophilic?

Direct immobilization of the multimeric protein to the surface can be attained by reacting an amino acid on the protein with the surface itself or by disposing a coating with reactive groups on the channel surface. Different amino acids in a protein biopolymer structure are used for covalent attachment. For example, the most common method of attachment of proteins to surfaces is through the amine groups of lysine residues. The thiol groups of cysteine molecules, as well as the carboxylic groups of aspartic acid and glutamic acid are also employed. The surface of the substrate usually contains groups that are reactive and can directly be used for attachment to the multimeric biopolymer. In some cases, however, the surface of the substrate needs to be activated to introduce reactive groups for attachment. A number of surface modifying reactions are commonly employed, and include the use of diazo, glutaraldehyde, cyanogen bromide (CNBr), carbodiimide, epoxide, and 2-fluoro-1-methylpyridinium tosylate (FMP). Upon activation of the substrate surface, the multimeric biopolymer is then directly attached through the amine, thiol, or carboxylic groups present in the multimeric biopolymer. Additionally, multimeric biopolymers polymer may be attached to the substrate by introducing complementary affinity pairs into both polymers. For example, the biotin/streptavidin system mentioned in the case of the immobilization of the oligonucleotides to the redox surface is also employed here. Biotin and streptavidin can be attached to the multimer and substrate}by well-established chemical/biochemical protocols. The biotin/streptavidin system is not the only one suitable for this type of attachment, and other types of affinity pairs can also be employed.

The polymeric aptamers can also be attached to a surface of the substrate by one of the many methods found in the literature to attach nucleic acids.

In a further embodiment, the multimeric biopolymer is blended or attached to a redox polymer which is in electrical contact with a conductor, e.g. a metal or carbon electrode, so that protons generated at the redox polymer through electrochemical action are released closer to the multimeric biopolymer to affect the three-dimensional structure thereof. Generally speaking, it is preferable to have more than one means of controlling the actions of a device. This is especially true in the case of medical devices, where the need to ensure safety and efficacy inevitable requires some backup control system that is externally accessible and able to override the chemical control system if the device is not functioning properly or its actions are no longer appropriate to the needs of the patient. Such a backup system for the present devices may be illustrated through the rigid channel example, with the addition of an element of electronic control through use of redox polymer. The main benefit of this approach is the ability to use the external electrochemical potential to override the chemical actuation. The overriding can result in either a permanent change in the structure of the multimeric biopolymer (desirable in cases where the system needs to be shut off, for example when a device begins to fail), or in a reversible change of the three-dimensional structure of the multimeric biopolymer. The latter is important when a binding event needs to be reversed for resetting the device. An additional benefit of the “wired” system is the speed by which this electrochemically-induced changes can be imposed on the multimeric biopolymer/redox polymer blend.

The electronic backbone is typically a redox polymer such as polypyrrole, polyaniline, polythiophene, etc. The redox polymer may be deposited by electrodeposition from a solution comprising the precursors thereof onto a conductor surface such as a patterned metal electrode thereby confining the actuator onto the conductive parts of a MEMS or NEMS structure only. The multimeric biopolymer may be lithographically patterned silk screened or drop delivered onto the metal electrode. Preferably, the device further comprises a small battery, a microprocessor (ideally incorporating telemetry), and a storage chamber for holding substance which is dispensed when an analyte binds to the multimeric biopolymer. For devices which are used to deliver thereapeutic compounds such as for example a drug, it is preferred that the device be implantable and be comprised or coated with a biocompatible substance.

In a further embodiment, the device further comprises an override system which comprises a hydrogel/redox polymer blend instead of just a redox polymer. This allows coupling of the binding event with swelling/shrinking of the hydrogel while maintaining the override of the chemical actuation by an external chemical potential. The redox polymer may be seen as a conductive electrode extending throughout the hydrogel. The major benefit is that ionic changes induced by a potential change on the metal electrode are now distributed throughout the hydrogel making for a faster response of this mixed conductor system. The mechanism of swelling and shrinking remains the same as with the hydrogel on a metal electrode (see FIG. 1) except that the effect is faster and can permeate through a thicker layer of hydrogel. Moreover the effect is not necessarily based on a pH change. For example, the effect may be based on water uptake by the hydrogel.

The redox polymer can be electrodeposited on the conductor with the gel film already in place. A hydrogel is permeable to the monomers of a redox polymer so the hydrogel may be placed over the metal electrode and with the electrode biased properly the monomer polymerizes within the overlaying hydrogel. Alternatively the hydrogel and the redox monomers may be mixed beforehand and polymerized in situ on the metal electrode. The redox-polymer/hydrogel blend may then be further modified chemically by incorporating a multimeric biopolymer using any of the chemical attachment schemes discussed above.

### EXAMPLES

The invention may be better understood by reference to the following examples, which serve to illustrate but not to limit the present invention.

### Example 1

Making of a Calmodulin Dimer Protein

Calmodulin is a calcium-binding protein that also binds phenothiazines (see FIG. 1). When calmodulin binds calcium, it undergoes a conformational change. This conformational change allows calmodulin to interact with calmodulin binding proteins, peptides, and drugs such as trifluoropiperazine and phenothiazine. Such a conformational change will be larger when single calmodulin molecules are linked or fused together to yield a polymeric calmodulin molecule comprised of at least two single calmodulin molecules. This example describes preparation of a calmodulin dimer, a single molecule comprised of two single calmodulin molecules.

A calmodulin dimer protein is made by fusing two calmodulin-encoding genes together, end-to-end. Such gene fusion techniques are well known to those experienced in the art. The calmodulin dimer fusion gene is then cloned into a plasmid that will allow expression of the gene in bacteria. The plasmid is used to transform Escherichia coli and bacterial colonies that contain the plasmid are selected. The transformed E. coli are grown and the calmodulin dimer protein is isolated from the cells using standard protein purification techniques well known to those skilled in the art. Calmodulin is then purified using a phenothiazine affinity column, to which calmodulin binds in the presence of calcium, and is eluted with an EGTA-containing buffer (Hentz and Daunert, 1996, Anal Chem, 68:3939-44.; Hentz, et al., 1996, Anal Chem, 68:1550-5.).

### Example 2

Comparison of Ca²⁺ Conformation Changes in Calmodulin Monomer and Dimer Proteins Using the Fluorescence Assay

In order to measure the extent of conformation change in calmodulin molecules in response to calcium and phenothiazine binding, a system has been developed in which a fluorophore is conjugated to a cysteine residue that had been inserted at amino acid 109 of wild type calmodulin using methods well known to those skilled in the art. This calmodulin mutant was called CaM109. The fluorescence of the fluorophore increases as the conformation of calmodulin changes in the presence of calcium.

Since wild-type calmodulin contains no cysteines, the addition of a cysteine to calmodulin at a desired position within the protein, allows for labeling of the protein at this position. Such labeling was done using a thiol-reactive fluorescent label called N-[2-(1-maleimidyl)ethyl]-7-(diethylamino)coumarin-3-carboxamide, or MDCC. MDCC was synthesized using methods known to the literature (Corrie, 1990, J. Chem. Soc. Perkin Trans. 1:2151-2152; Corrie, 1994, J. Chem. Soc. Perkin Trans. 1:2975-2982).

The fluorescence response of MDCC-labeled, CaM109 molecules was recorded in the absence and presence of 3×10⁶ M Ca²⁺. The Ca²⁺ concentration was controlled by EGTA at pH 8.0, and the free Ca²⁺ concentrations were calculated using the software program Chelator (Haugland, 1996, Handbook of Fluorescent Probes and Research Chemicals, 6th edition, Molecular Probes, Eugene, Oreg., p. 52). The results showed that the fluorescence intensity of the molecules increased 90% as compared to calmodulin molecules to which calcium had not been added. In addition, when phenothiazine was added to labeled calmodulin molecules that that had already bound calcium, fluorescence was quenched 100%.

Calmodulin monomer and dimer proteins are made by expression in E. coli and purified as described in Example 1. These proteins are then separately labeled with MDCC as described in Example 2. The MDCC-labeled calmodulin monomer and dimers proteins are then recorded in the absence and presence of 3×10⁶ M Ca²⁺, as described in Example 2. The results show that increase in fluorescence of the calmodulin dimer protein is greater than the increase in fluorescence of the calmodulin monomer protein.

It should be understood that the preceding is merely a detailed description of preferred embodiments. It therefore should be apparent to those of ordinary skill in the art that various modifications and equivalents can be made without departing from the spirit and scope of the invention. All references, patents and patent publications that are identified in this application are incorporated in their entirety herein by reference. The specific examples presented below are illustrative only and is not intended to limit the scope of the invention described herein.

What is claimed is:

1. A synthetic multimeric biopolymer comprising a plurality of monomeric units selected from the group consisting of proteins, polypeptides, nucleic acids, peptide nucleic acids, and combinations thereof;
wherein said monomeric units are the same or different;
wherein at least one of said plurality of monomeric units comprises a binding region for an analyte; and
wherein said multimeric biopolymer changes its three-dimensional conformation in response to binding of the analyte to said binding region.

2. The biopolymer of claim 1 wherein the monomeric unit which comprises the binding region for the analyte is a protein or polypeptide.

3. The biopolymer of claim 1 wherein the monomeric unit which comprises the binding region for the analyte is a nucleic acid molecule.

4. The biopolymer of claim 1 wherein the monomeric unit which comprises the binding region for the analyte is an aptamer.

5. The biopolymer of claim 1 wherein the monomeric unit which comprises the binding region for the analyte is a peptide nucleic acid.

6. The biopolymer of claim 1 wherein said biopolymer comprises a protein, polypeptide, or aptamer that changes its three-dimensional conformation in response to binding of a proton to or a release of a proton from said binding region.

7. The biopolymer of claim 1 wherein said biopolymer comprises an enzyme that catalyzes a biochemical reaction which results in the formation of protons or hydroxide ions when said enzyme binds to said analyte.

8. The biopolymer of claim 1 wherein said biopolymer comprises
(a) a protein or polypeptide that changes its three-dimensional conformation in response to binding of a proton or a hydroxide to said binding region, and
(b) a protein or polypeptide that catalyzes a biochemical reaction which results in the formation of protons or hydroxide ions when said protein or said polypeptide binds to said analyte.

9. The biopolymer of claim 1 wherein said biopolymer comprises a plurality of proteins or polypeptides or a plurality of aptamers.

10. The biopolymer of claim 1 wherein said biopolymer comprises from 2 to 10 monomeric units.

11. The biopolymer of claim 1 wherein the analyte is selected from the group consisting of a sugar, a protein, a peptide, a nucleic acid, a hormone, a vitamin, a co-factor, an anion and a cation.

12. A synthetic multimeric biopolymer comprising a plurality of monomeric units selected from the group consisting of a protein, a polypeptide, a nucleic acid, and a peptide nucleic acid, wherein said monomeric units are the same or different,
wherein at least one of said plurality of monomeric units comprises a binding region for an analyte, and
wherein binding of the analyte to said binding region results in the formation of protons or the transmission of a detectable signal by the multimeric polymer.

13. The biopolymer of claim 12 wherein said biopolymer comprises a protein or polypeptide that catalyzes a biochemical reaction which results in the formation of protons or hydroxides when said protein or said polypeptide binds to said analyte

14. The biopolymer of claim 12 wherein said biopolymer comprises a monomeric unit that transmits a detectable signal selected from the group consisting of a fluorescent signal, an optical signal, an electrochemical signal, a pressure change, a dielectric constant change, a mass change, a volume change, and a temperature change in response to binding of the analyte to said binding region.

15. A device for dispensing a substance in response to an analyte, comprising:
(a) a substrate having at least one delivery chamber which contains the substance; and
(b) a multimeric biopolymer which is disposed in a channel in communication with said at least one delivery chamber or in an opening to said at least one delivery chamber;
wherein said multimeric biopolymer compres a plurality of monomeric units selected from the group consisting of proteins, polypeptides, nucleic acids, peptide nucleic acids, and combinations thereof;
wherein said monomeric units are the same or different;
wherein at least one of said plurality of monomeric units comprises a binding region for an analyte; and
wherein said multimeric biopolymer changes its three-dimensional conformation in response to binding of the analyte to said binding region; and
wherein changes in the three dimensional conformation of the multimeric biopolymer regulate the opening and closing of the channel or the delivery chamber opening.

16. The device of claim 15, wherein said device is a microchip, a nanochip, a nanovial, a microvial, microchannel, nanochannel, a microelectromechanical system (MEMS), or a nanoelectromechanical system (NEMS).

17. The device of claim 15, wherein said substance is a therapeutic material.

18. The device of claim 15, wherein the substrate comprises a porous or nanoporous material.

19. The device of claim 18, wherein said porous or nanoporous material is alumina or zeolite or titania, or silica, or zirconia.

20. The device of claim 15 wherein the monomeric unit which comprises the binding region is a protein or polypeptide.

21. The device of claim 15 wherein the monomeric unit which comprises the binding region is a nucleic acid molecule.

22. The device of claim 15, wherein the monomeric unit which comprises the binding region is an aptamer.

23. The device of claim 15, wherein said multimeric biopolymer is covalently bound to a surface which defines the channel or to a coating which is disposed on a surface which defines the channel.

24. The device of claim 15 wherein the coating is a hydrophilic substance.

25. The device of claim 15 wherein a decrease in the size of the multimeric biopolymer opens the channel or opening.

26. The device of claim 1 wherein said multimeric biopolymer is in contact with a moveable member adapted to move from a position away from said opening and a position covering said opening; and wherein the change in the three dimensional conformation of said multimeric biopolymer results in movement of said movable member.

27. The device of claim 26 wherein the moveable member comprises a hydrogel or a rigid substance.

28. The device of claim 15 further comprising an electronically conducting redox polymer in contact with said multimeric biopolymer;
wherein the multimeric biopolymer comprises a first binding region for binding to an analyte which is not a proton and second binding region for binding to a proton;
wherein binding to the analyte which is not a proton to the multimeric biopolymer and binding of the proton to the multimeric polymer have opposite effects on the three-dimensional change in conformation of the multimeric biopolymer; and
wherein application of an electrical potential to said redox polymer results in the accumulation of protons in the microenvironment of the multimeric biopolymer, or
removal of protons from the microenvironment of the multimeric biopolymer.

29. The device of claim 28 wherein the redox polymer is blended with or covalently bonded to the multimeric biopolymer.

30. The device of claim 28 wherein the redox polymer is in contact with a electron conductor.

30. The device of claim 29 wherein the redox polymer is deposited on a metal electrode.

31. The device of claim 28 wherein the redox polymer is selected from the group consisting of consisting of polyanilines, polypyrroles, polythiophenes, polyindoles, and mixtures thereof.

32. The device of claim 15 further comprising a redox polymer in proximity to said multimeric biopolymer for generating protons or hydroxide ions near the multimeric biopolymer and reversing the reaction which results from binding of the analyte to the multimeric biopolymer, wherein said redox polymer is attached to an electrode.

33. The device of claim 15 further comprising a material capable of changing its shape disposed in the channel or the opening of the delivery chamber,
said material comprising an electronically conducting redox polymer and a hydrogel;
wherein application of an electrical potential to said redox polymer results in a change in shape of the hydrogel.

34. The device of claim 33 wherein said hydrogel is selected from the group consisting of polyhydroxyethylmethacrylates.

35. The device of claim 33 wherein application of a redox polymer causes said material capable of changing its shape to shrink, thereby opening said at least one delivery chamber or channel, or causes said material capable of changing its shape to swell, thereby closing said at least one delivery chamber or channel.

36. The device of claim 33 wherein application of a redox polymer causes said material capable of changing its shape to swell, thereby opening said at least one delivery chamber or channel or causes said material capable of changing its shape to shrink, thereby opening said at least one delivery chamber or channel.

37. The device of claim 33, wherein said material capable of changing its shape is in contact with an electrode.

38. A device for detecting the presence of an analyte in a medium, said device comprising
a) a substrate, and
b) a multimeric biopolymer disposed on said substrate
said multimeric biopolymer comprising a plurality of monomeric units selected from the group consisting of proteins, polypeptides, nucleic acids, peptide nucleic acids, and combinations thereof;
wherein said monomeric units are the same or different,
wherein at least one of said plurality of monomeric units comprises a binding
region for said analyte, and
wherein said multimeric biopolymer emits a detectable signal when said analyte binds to said binding region.

39. The device of claim 38 wherein the detectable signal is selected from the group consisting a fluorescent signal, an optical signal, an electrochemical signal, a pressure change, a dielectric constant change, a mass change, a volume change, and a temperature change.

40. A device for dispensing a substance, comprising dispensing a substance in response to an analyte, comprising:
(a) a substrate having at least one delivery chamber which contains the substance; and
(b) a multimeric biopolymer and a hydrogel,
wherein said multimeric biopolymer and said hydrogel are disposed in a channel in communication with said at least one delivery chamber or in an opening to said at least one delivery chamber;
wherein said multimeric biopolymer compres a plurality of monomeric units selected from the group consisting of proteins, polypeptides, nucleic acids, peptide nucleic acids, and combinations thereof;
wherein said monomeric units are the same or different;
wherein at least one of said plurality of monomeric units comprises a binding region for an analyte; and
wherein said multimeric biopolymer releases a charged ion in response to binding of the analyte to said binding region; and
wherein release of the charged ion cause a change in the pH of the microenvironment of the hydrogel, thereby causing the hydrogel to shrink or swell.

41. A synthetic multimeric biopolymer comprising a plurality of monomeric units selected from the group consisting of proteins, polypeptides, nucleic acids, peptide nucleic acids, and combinations thereof;
wherein said monomeric units are the same or different;
wherein at least one of said plurality of monomeric units comprises a binding region for an analyte; and
wherein said multimeric biopolymer amplifies changes in its three-dimensional conformation in response to binding of the analyte to said binding region.
